(19) United States
(12) Patent Application Publication
(10) Pub. No.: US 2026/0245688 A1
(43) Pub. Date: Aug. 20, 2026

(54) FETAL ALIGNMENT RECOMMENDATIONS

(71) Applicant: GE Precision Healthcare LLC, Waukesha, WI (US)

(72) Inventors: Rakhi Daniel, Arlington Heights, IL (US); Pavithra Vinay, Arlington Heights, IL (US); Arun Polepaka, South Barrington, IL (US); Srikanth Melugiri, Chicago, IL (US)

(73) Assignee: GE Precision Healthcare LLC, Waukesha, WI (US)

(21) Appl. No.: 19/054,037

(22) Filed: Feb. 14, 2025

(51) Int. Cl. G16H 20/00; A61B 5/00; G16H 70/20

(52) U.S. Cl. CPC G16H 20/00; A61B 5/4362; A61B 5/742; G16H 70/20

(57) ABSTRACT

A system includes a processor and a memory. The memory includes instructions executable by the processor to generate fetal alignment recommendations by querying a database of fetal alignment recommendations. Further, querying is based on maternal health parameters for an expectant mother in labor, fetal health parameters for a fetus of the expectant mother, a position of the fetus, a presentation of the fetus, and a descent of the fetus. Additionally, the instructions are executable by the processor to randomly selecting a recommendation from multiple fetal alignment recommendations, and present the recommendation in view of a user interface of a fetal monitoring application.

### BACKGROUND

The present disclosure generally relates to fetal alignment, and more particularly fetal alignment recommendations.

In clinical applications, healthcare professionals may monitor the well-being of an expectant mother and her fetus during term and pre-term labor. Monitoring may include, for example, the use of electrode patches to track maternal and fetal heart rate, uterine activity, and specific percentage of oxygen concentration (SpO2) in the blood. Additionally, monitoring may include ultrasound and/or manual palpitation to determine the position and presentation of the fetus.

### SUMMARY

This Summary is provided to introduce a selection of concepts that are further described below in the Detailed Description. This Summary is not intended to identify key or essential features of the claimed subject matter, nor is it intended to be used as an aid in limiting the scope of the claimed subject matter.

A system includes a processor and a memory. The memory includes instructions executable by the processor to generate fetal alignment recommendations by querying a database of fetal alignment recommendations. Further, querying is based on maternal health parameters for an expectant mother in labor, fetal health parameters for a fetus of the expectant mother, a position of the fetus, a presentation of the fetus, and a descent of the fetus. Additionally, the instructions are executable by the processor to randomly selecting a recommendation from multiple fetal alignment recommendations, and present the recommendation in view of a user interface of a fetal monitoring application.

In one embodiment, querying includes using a machine learning model trained to provide the first plurality of fetal alignment recommendations based on the plurality of maternal health parameters, the plurality of fetal health parameters, the position of the fetus, the presentation of the fetus, and the descent of the fetus.

In another embodiment, the instructions are executable by the processor to remove one or more previous fetal alignment recommendations that the expectant mother has attempted before randomly selecting the recommendation.

In another embodiment, querying is further based on a result of implementing the one or more previous fetal alignment recommendations.

In another embodiment, querying is further based on an instruction from a healthcare provider for the expectant mother.

In another embodiment, querying is further based on a position of a placenta.

In another embodiment, the fetal monitoring application determines the plurality of maternal health parameters and the plurality of fetal health parameters.

In another embodiment, the instructions are executable by the processor to identify a media source that provides a visual representation of the recommendation, and provide the visual representation by accessing the media source, and displaying the visual representation in view of the user interface of the fetal monitoring application.

A method includes generating fetal alignment recommendations by querying a database of fetal alignment recommendations. Querying is based on maternal health parameters for an expectant mother in labor, fetal health parameters for a fetus of the expectant mother, a position of the fetus, a presentation of the fetus, and a descent of the fetus. The method also includes generating remaining fetal alignment recommendations by removing, from the generated fetal alignment recommendations, one or more previous fetal alignment recommendations that the expectant mother has attempted. Additionally, the method includes randomly selecting a recommendation from the remaining fetal alignment recommendations. Further, the method includes presenting the recommendation in view of a user interface of a fetal monitoring application.

In one embodiment, querying involves using a machine learning model trained to provide the fetal alignment recommendations based on the maternal health parameters, the fetal health parameters, the position of the fetus, the presentation of the fetus, and the descent of the fetus.

In another embodiment, querying is further based on an instruction from a healthcare provider for the expectant mother.

In another embodiment, querying is further based on a position of a placenta.

In another embodiment, querying is further based on a result of implementing the one or more previous fetal alignment recommendations.

In another embodiment, the fetal monitoring application determines the maternal health parameters and the fetal health parameters.

Another method includes identifying a media source that provides a visual representation of the recommendation. Additionally, the method includes providing the visual representation by accessing the media source, and displaying the visual representation in view of the user interface of the fetal monitoring application.

A computer readable medium includes instructions executable by a processor to generate fetal alignment recommendations by querying a database of fetal alignment recommendations. Querying is based on maternal health parameters for an expectant mother in labor, fetal health parameters for a fetus of the expectant mother, a position of the fetus, a presentation of the fetus, and a descent of the fetus. Additionally, the instructions are executable by the processor to generate remaining fetal alignment recommendations by removing, from the fetal alignment recommendations, one or more previous fetal alignment recommendations that the expectant mother has attempted. Further, the instructions are executable by the processor to randomly select a recommendation from the remaining fetal alignment recommendations, and present the recommendation in view of a user interface of a fetal monitoring application.

In one embodiment, querying includes using a machine learning model trained to provide the fetal alignment recommendations based on the maternal health parameters, the fetal health parameters, the position of the fetus, the presentation of the fetus, and the descent of the fetus.

In another embodiment, querying is further based on a result of implementing the one or more previous fetal alignment recommendations.

In another embodiment, the fetal monitoring application determines the maternal health parameters and the fetal health parameters.

In another embodiment, the instructions are executable by the processor to identify a media source that provides a visual representation of the recommendation, and provide the visual representation by accessing the media source, and displaying the visual representation in view of the user interface of the fetal monitoring application.

Various other features, objects, and advantages of the invention will be made apparent from the following description taken together with the drawings.

### BRIEF DESCRIPTION OF THE DRAWINGS

The present disclosure is described with reference to the following Figures.

FIG. 1 is a diagram of an example system for fetal alignment recommendations, according to one embodiment of the present disclosure.

FIG. 2 is a diagram of an example monitoring user interface (UI) and an example recommendation UI for fetal alignment recommendations, according to one embodiment of the present disclosure.

FIG. 3A is a diagram of an example monitoring UI and example recommendation UI for fetal alignment recommendations, according to one embodiment of the present disclosure.

FIG. 3B is a diagram of an example monitoring UI and example recommendation UI for fetal alignment recommendations, according to one embodiment of the present disclosure.

FIG. 3C is a diagram of an example monitoring UI and example recommendation UI for fetal alignment recommendations, according to one embodiment of the present disclosure.

FIG. 3D is a diagram of an example monitoring UI and example recommendation UI for fetal alignment recommendations, according to one embodiment of the present disclosure.

FIG. 4 is a flow chart of an example method for fetal alignment recommendations, according to one embodiment of the present disclosure.

FIG. 5 is a diagram of an example fetal alignment recommendation manager, according to one embodiment of the present disclosure.

### DETAILED DESCRIPTION

In the present description, certain terms have been used for brevity, clarity and understanding. No unnecessary limitations are to be inferred therefrom beyond the requirement of the prior art because such terms are used for descriptive purposes only and are intended to be broadly construed.

As used herein, unless otherwise limited or defined, discussion of particular directions is provided by example only, with regard to particular embodiments or relevant illustrations. For example, discussion of "top," "bottom," "front," "rear," "left," "right," "horizontal," "vertical," and "longitudinal" features and/or relative motion, e.g., movement "up" and "down," is generally intended as a description only of the orientation of such features relative to a reference frame of a particular example or illustration. Correspondingly, for example, a "top" feature may sometimes be disposed below a "bottom" feature (and so on), in some arrangements or embodiments. Additionally, or alternatively, embodiments may be arranged in a different orientation such that "top" and "bottom" features are arranged horizontally relative to each other, for example in a "left-to-right" orientation.

The use herein of the terms "including," "comprising," or "having," and variations thereof, is meant to encompass the elements listed thereafter and equivalents thereof, as well as additional elements. Embodiments recited as "including," "comprising," or "having" certain elements are also contemplated as "consisting essentially of" and "consisting of" those certain elements.

The inventors have recognized problems with current techniques for fetal positioning during term and pre-term labor. Fetal positioning may be useful to alleviate the expectant mother’s discomfort, aid in labor progression, and increase the likelihood of a smooth delivery. However, multiple factors may affect fetal positioning, such as, the maternal body mass index (BMI), gestational age, placental position, pitocin use, and mobility limitations.

In view of the foregoing problems and challenges recognized by the inventors through their extensive research and experience in the field of maternal and fetal monitoring, the inventors have developed the disclosed method and system for fetal alignment recommendations. According to some embodiments of the present disclosure, the fetal alignment recommendation manager integrates with a fetal monitoring application. Additionally, the fetal alignment recommendation manager may make recommendations for the expectant mother to change her body position to change the fetal position, as described above. Further, the fetal alignment recommendation manager may present the positioning recommendations on the same screen as the fetal monitoring user interface. In this way, the fetal alignment recommendation manager may enable a healthcare provider or other clinician to access fetal alignment recommendations without looking away from the fetal monitoring user interface. Further, by integrating with the fetal monitoring application, the fetal alignment recommendation manager may acquire data relevant to fetal positioning from the fetal monitoring application, and make recommendations based on the acquired data. According to some embodiments of the present disclosure, the fetal alignment recommendation manager may provide a user interface for entering data that is relevant to fetal alignment recommendations. Additionally, or alternatively, by integrating with the fetal monitoring application, the fetal alignment recommendation manager may acquire data relevant to fetal positioning from the fetal monitoring application, and make recommendations based on the acquired data. Further, the fetal alignment recommendation manager may interface with other applications having data that is relevant to fetal alignment recommendations. For example, the fetal alignment recommendation manager may interface with administrative software that tracks patient data, such as, body mass index, age, gestational age, and the like. In these ways, the fetal alignment recommendation manager may enable a healthcare provider or other clinician to aid an expectant mother in term or pre-term labor with fetal alignment recommendations that may alleviate the patient’s discomfort, aid in labor progression, and improve the likelihood of a health delivery.

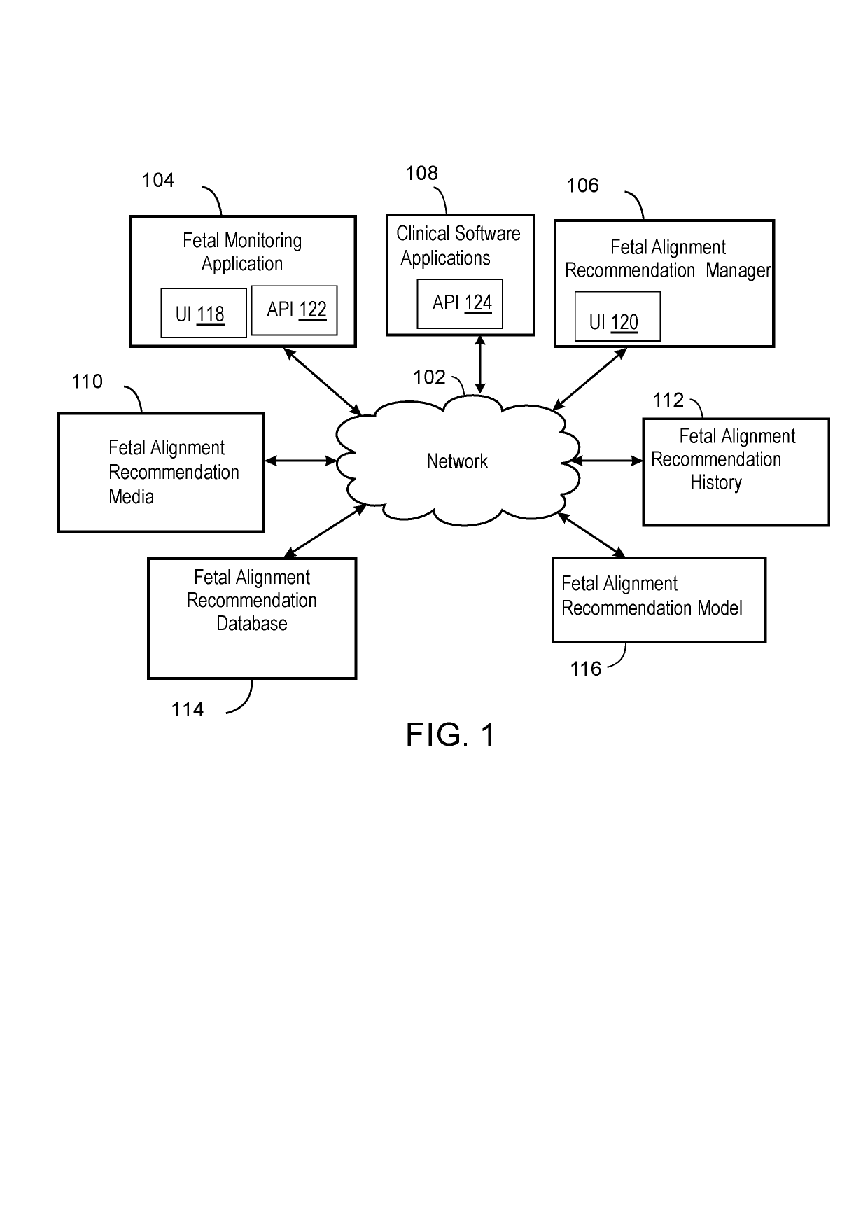

FIG. 1 is a diagram of an example system 100 for fetal alignment recommendations, according to one embodiment of the present disclosure. The system 100 includes a network 102, fetal monitoring application 104, fetal alignment recommendation manager 106, clinical software applications 108, fetal alignment recommendation media 110, fetal alignment recommendation history 112, fetal alignment recommendation database 114, and fetal alignment recommendation model 116. The network 102 may be one or more computer communication networks, such as a local area network, wide area network, and the like, that is configured to exchange messaging between the fetal monitoring application 104, fetal alignment recommendation manager 106, clinical software applications 108, fetal alignment recommendation media 110, fetal alignment recommendation history 112, fetal alignment recommendation database 114, and fetal alignment recommendation model 116. In one embodiment of the present disclosure, the network 102 may be the Internet.

The fetal monitoring application 104 may be software that receives signals from clinical devices, such as electrode patches, ultrasound transducers, specific percentage oxygen saturation trackers, and the like, and presents the information in a monitoring user interface (UI) 118, on a display device. The display device may be a fetal monitor device, a computer monitor, mobile display device, and the like.

The fetal alignment recommendation manager 106 may be configured to make recommendations for the expectant mother to change her body position based on parameters, such as, maternal BMI, gestational age, mobility limitations, pitocin administration, fetal position, fetal presentation, fetal descent, cervical changes, and the like. The fetal alignment recommendation manager 106 may include a recommendation user interface (UI) 120. The fetal alignment recommendation manager 106 may display the recommendation UI 120 along with the monitoring UI 118 (e.g., as a pop-up window), thus enabling a healthcare provider or other clinician to provide inputs to the fetal alignment recommendation manager 106 without impeding their ability to continue monitoring the data on the monitoring UI 118. According to some embodiments of the present disclosure, the recommendation UI 120 may include an input screen that a healthcare professional or other clinician may use to enter the fetal alignment recommendation parameters, such as maternal BMI, gestational age, and the like. In some cases, the monitoring UI 118 may include fetal alignment recommendation relevant data, which may be queried for on the input screen of the recommendation UI 120. Thus, positioning the recommendation UI 120 within the monitoring UI 118 may enable the healthcare professional or other clinician to determine some of the query entries without looking away from the display.

Additionally, or alternatively, the fetal alignment recommendation manager 106 may interface with the fetal monitoring application 104 to acquire fetal alignment recommendation parameters. More specifically, the fetal monitoring application 104 may include a monitoring API 122 that the fetal alignment recommendation manager 106 uses to query for these parameters. Additionally, the system 100 may include clinical software applications 108, such as, an admission discharge transfer (ADT) system. An ADT system is a software application that a hospital or other healthcare facility may use to track patients while in the care of the facility. Clinical software applications 108 may have, in addition to other administrative data, access to patient data that is relevant to fetal alignment recommendations. Accordingly, the fetal alignment recommendation manager 106 may interface with the clinical software applications to acquire this data. More specifically, the clinical software applications 108 may include a clinical API 124. Accordingly, the fetal alignment recommendation manager 106 may query the clinical API 124 for one or more fetal alignment recommendation parameters.

Further, the recommendation UI 120 may include an output screen that displays fetal alignment recommendations and related data. Advantageously, the fetal alignment recommendation manager 106 may display this recommendation and related data with the monitoring UI 118, thus providing the healthcare professional or other clinician continued access to the output of the fetal monitoring application 104 while viewing the fetal alignment recommendation and related data. Additionally, the fetal alignment recommendation manager 106 may use the recommendation UI 120 to display photographic and/or video demonstrations of the recommended fetal alignment positions. More specifically, the fetal alignment recommendation manager 106 may retrieve a photograph and/or video demonstrating an example of the recommendation from fetal alignment recommendation media 110. The fetal alignment recommendation media 110 may be locally stored, e.g., on the local network or monitoring device, or in Internet-accessible media sources. In some cases, a fetal alignment recommendation may not provide the expectant mother relief from discomfort, and/or may not aid in the labor progression. In other cases, the fetal alignment recommendation may provide temporary relief and/or aid. As such, the fetal alignment recommendation manager 106 may provide an additional recommendation in response to a request from the healthcare provider or other clinician. Further, the fetal alignment recommendation manager 106 may generate a fetal alignment recommendation history 112. The fetal alignment recommendation history 112 may include each of the recommendations, whether or not the expectant mother tried the recommendation, the time of the attempt, how long the recommended position is held, the fetal alignment recommendation parameters at the time of the recommendation, and the result of the attempt. The result of the attempt may indicate whether the labor advances, whether the expectant mother’s pain is alleviated, physiological changes (e.g., maternal and/or fetal heart rate) of the expectant mother and/or fetus, and the like. Additionally, the fetal alignment recommendation history 112 may include, the outcome of the labor for the patient, e.g., a vaginal birth, caesarean section, successful birth, and the like.

The fetal alignment recommendation database 114 may be a datastore of recommendations for positional changes for the expectant mother during labor. These fetal alignment recommendations may include a label and textual description of the positional change. Additionally, the fetal alignment recommendation database 114 may include links to fetal alignment recommendation media 110 that provide a pictorial and/or video demonstration of the fetal alignment recommendation.

The fetal alignment recommendation model 116 may be a machine learning model that is trained to select one or more fetal alignment recommendations for the expectant mother based on the fetal alignment recommendation parameters described above. The fetal alignment recommendation model 116 may be trained using fetal alignment recommendation history 112, which includes the fetal alignment recommendation parameters for each fetal alignment recommendation, the result of the attempt, and the outcome of the labor. Additionally, the fetal alignment recommendation model 116 may be trained based on combinations and/or sequences of prior fetal alignment recommendations, and whether these combinations and/or sequences led to positive or negative results and/or positive or negative labor outcomes. Accordingly, the result and the outcome of the labor may represent labels of the training data. As such, the fetal alignment recommendation model 116 may be trained to select fetal alignment recommendations to improve the likelihood of positive results and positive labor outcomes. For example, results such as advancing labor and alleviating pain may represent positive results. Conversely, results such as, no advance, and/or no pain relief may represent negative results. Further, labor outcomes, such as vaginal birth, and successful birth may represent positive outcomes. Conversely, caesarean birth, and/or malpresentation may represent negative outcomes. Malpresentation refers to when the vertex of the fetal head is not the part of the fetus closest to the pelvic inlet, which may complicate delivery, and thus result in negative labor outcomes.

According to some embodiments of the present disclosure, in response to a request for fetal alignment recommendations, the fetal alignment recommendation model 116 may provide the names and/or textual descriptions of one or more fetal alignment recommendations. Additionally, the fetal alignment recommendation model 116 may provide links to fetal alignment recommendation media 110 demonstrating the fetal alignment recommendations. According to some embodiments of the present disclosure, the fetal alignment recommendation model 116 may provide keys or other identifiers for retrieving the descriptions and/or fetal alignment recommendation media links from the fetal alignment recommendation database 114.

According to some embodiments of the present disclosure, the fetal alignment recommendation manager 106 may request one or more recommendations from the fetal alignment recommendation model 116 by providing the fetal alignment recommendation parameters of the expectant mother, and the fetal alignment recommendation history 112 for the current labor as inputs. When in receipt of multiple recommendations from the fetal alignment recommendation model 116, the fetal alignment recommendation manager 106 may randomly select one fetal alignment recommendation from the multiple fetal alignment recommendations. Further, in embodiments where the fetal alignment recommendation model 116 provides a key for the fetal alignment recommendation database 114, the fetal alignment recommendation manager 106 may retrieve the fetal alignment recommendation data, description, and/or fetal alignment recommendation media links from the fetal alignment recommendation database 114. Additionally, the fetal alignment recommendation manager 106 may present the fetal alignment recommendation in the recommendation UI 120. In the event that the fetal alignment recommendation model 116 provides one fetal alignment recommendation, the fetal alignment recommendation manager 106 may perform the above-described techniques for the single recommendation.

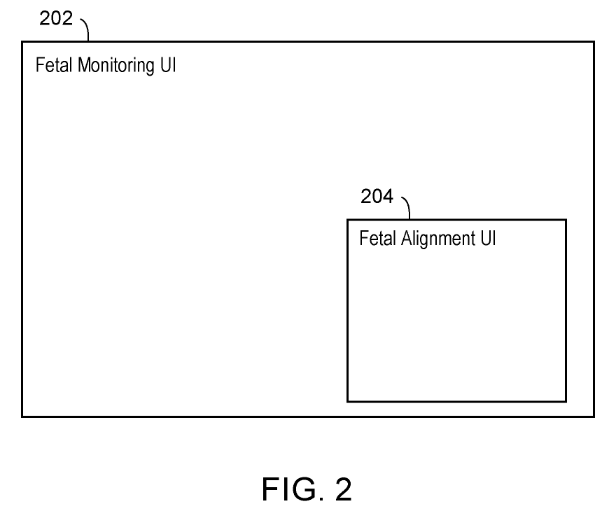

FIG. 2 is a diagram of an example monitoring UI 202 and an example recommendation UI 204 for fetal alignment recommendations, according to one embodiment of the present disclosure. The monitoring UI 202 may be an interface that provides physiological data about the expectant mother and fetus during labor. Such data may include maternal and fetal heart rates, uterine activity, blood pressure, SpO2, and the like. As stated previously, some embodiments of the present disclosure may display the recommendation UI 204 with, or on the same screen as the monitoring UI 202. Further, such embodiments may display the recommendation UI 204 in a manner that does not obstruct the view of the maternal and fetal waveform data and vitals on the monitoring UI 202. As such, the recommendation UI 204 may be a pop-up window that can open below the fetal strip, and/or in a separate tab from the monitoring UI 202. This separate tab may still include the view of the maternal and fetal waveform data and vitals. According to some embodiments of the present disclosure, the monitoring UI 202 may include a selection element 206, such as an on-screen button, that a healthcare professional or other clinician may engage to launch the fetal alignment recommendation manager 106, which may request fetal alignment recommendations from the fetal alignment recommendation model 116, and hence display the recommendation UI 204 with the recommendation, as described below.

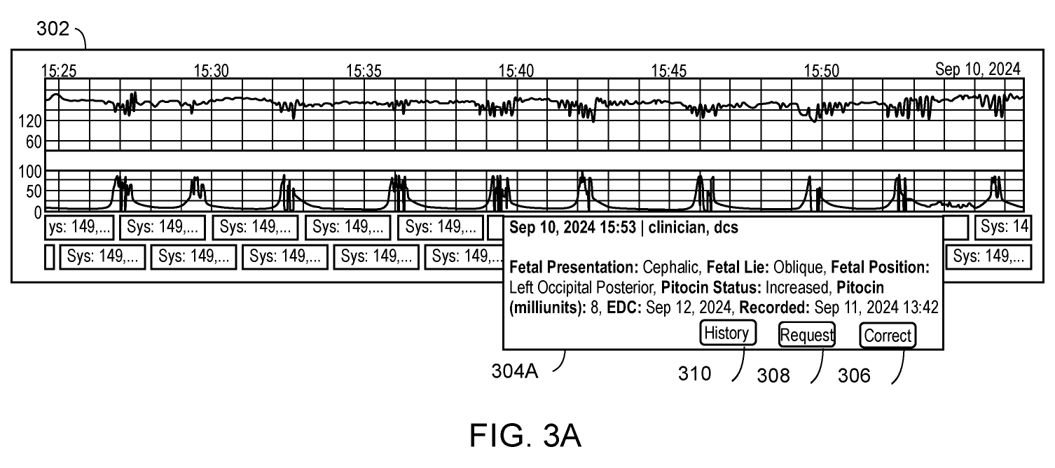

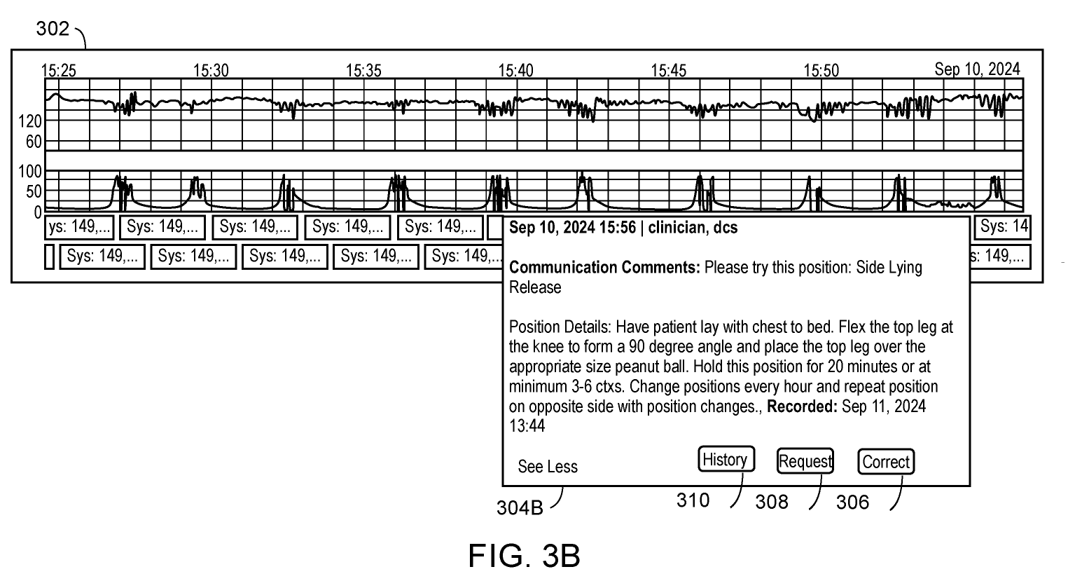

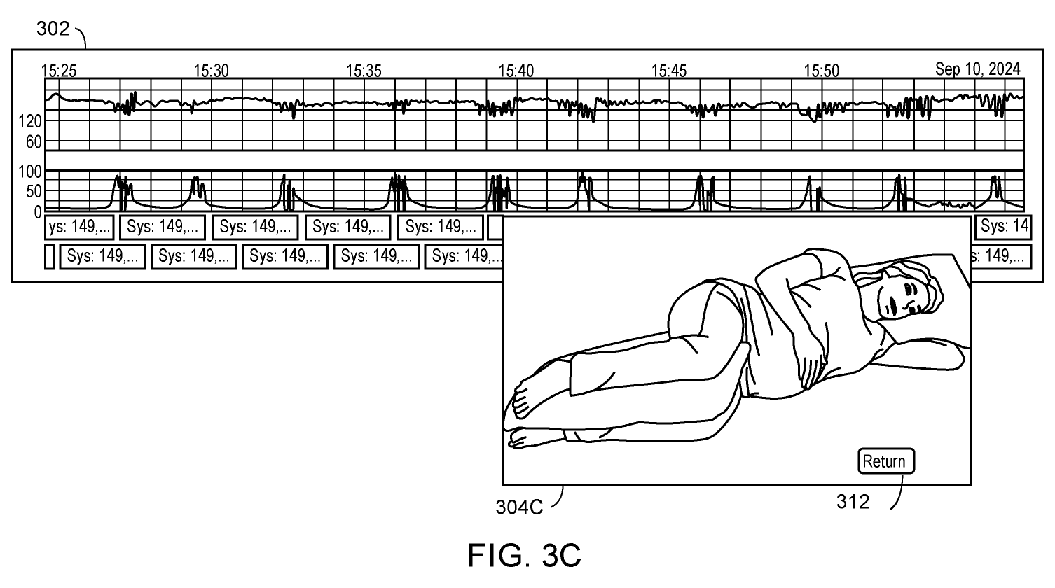

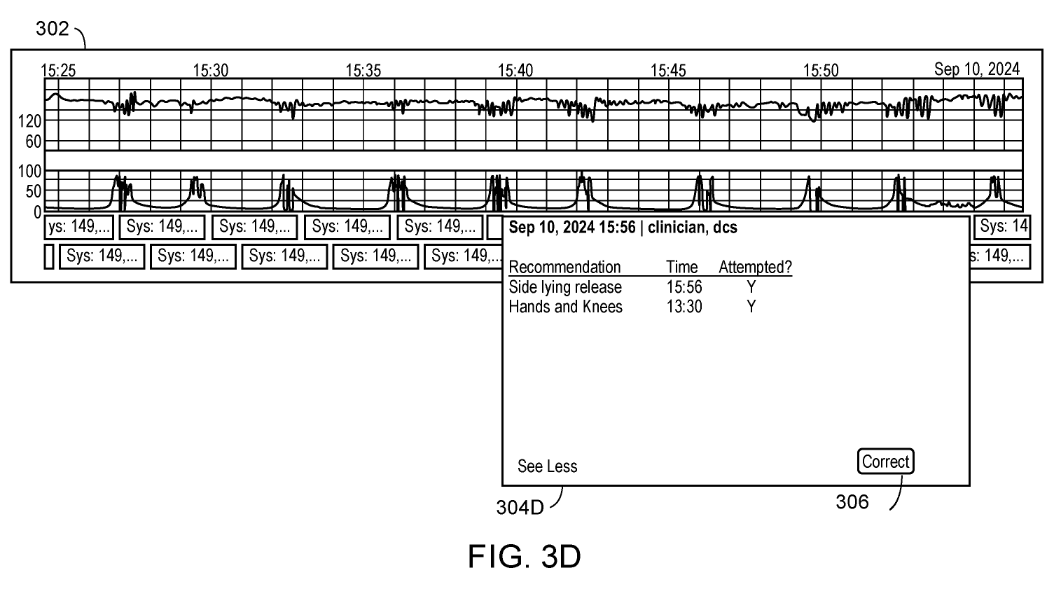

FIG. 3A is a diagram of an example monitoring UI 302 and example recommendation UI 304A for fetal alignment recommendations, according to one embodiment of the present disclosure. In this example, the monitoring UI 302 includes a graph (e.g., waveform) tracking maternal and fetal heart rates, uterine activity, and blood pressure, over time. Further, the recommendation UI 304A may represent a touch screen input interface for the healthcare professional or other clinician to provide additional fetal alignment recommendation parameters. In this example, the fields in the recommendation UI 304A include fetal presentation, fetal lie, fetal position, pitocin status, estimated date of confinement (EDC), and a timestamp for when the data is entered and/or acquired. As stated previously, the fetal alignment recommendation manager 106 may acquire one or more fetal alignment recommendation parameters from the respective APIs 122, 124 of the fetal monitoring application 104 and/or clinical software applications 108. In such embodiments, the fetal alignment recommendation manger 106 may pre-populate the recommendation UI 304A with the acquired data fields upon launch and/or as fetal alignment recommendation parameters update.

Additionally, the recommendation UI 304A may include action buttons for specific functions of the fetal alignment recommendation manager 106. In this example, the recommendation UI 304A includes a correct button 306, and a request button 308. In response to a selection, or other engagement, of the correct button 306, the fetal alignment recommendation manager 106 may provide the ability to edit the information in the recommendation UI 304A. For example, if the fetal presentation changes to breech, the healthcare professional may select the correct button 306. In response, the fetal alignment recommendation manager 106 may permit selection of a field to edit. Accordingly, the healthcare professional may select the fetal presentation field with a touch action, and update the field value to “breech” through the use of a keyboard. Other edits may also be possible according to some embodiments. For example, the healthcare professional may enter the results of a cervical exam, a doctor instruction, or any other data relevant to the fetal alignment recommendations through this use of the correct button 306.

Further, the recommendation UI 304A includes a request button 308. In response to a selection, or other engagement, of the request button 308, the fetal alignment recommendation manager 106 may make a request to the fetal alignment recommendation model 116 for one or more fetal alignment recommendations. Additionally, the fetal alignment recommendation manager 106 may randomly select from multiple fetal alignment recommendations provided by the fetal alignment recommendation model 116, and present the selected fetal alignment recommendation on an interface replacing the recommendation UI 304A, as described in greater detail below.

FIG. 3B is a diagram of the example monitoring UI 302 and example recommendation UI 304B for fetal alignment recommendations, according to one embodiment of the present disclosure. In this example, the fetal alignment recommendation manager 106 has populated the recommendation UI 304B with a fetal alignment recommendation, and a description of the fetal alignment recommendation. The fetal alignment recommendation is indicated as, “Communication Comments: Please try this position: Side Lying Release.” Further, the description is indicated in, “Position Details.” In this example, the name of the fetal alignment recommendation is underlined, indicating an active hyperlink. Accordingly, in response to a user selection of the hyperlink, the fetal alignment recommendation manager 106 may access a fetal alignment recommendation media source, retrieve fetal alignment recommendation media 110, and populate a new user interface, as described in greater detaial below.

Additionally, the recommendation UI 304B includes the request button 308, described with respect to FIG. 3A, and a history button 310. The history button 310 may be useful for viewing previous recommendations for the patient during labor, which is described in greater detail below, with respect to FIG. 3D. Further, in some cases, the expectant mother may not perform the fetal alignment recommendation. This may be due to various reasons, such as, a personal preference against, a mobility limitation not included in the fetal alignment recommendation parameters, and/or the advice of a healthcare professional, for example. In such a case, the healthcare professional may select the request button 308 again to request a different fetal alignment recommendation. Accordingly, the fetal alignment recommendation manager 106 may select another fetal alignment recommendation from the set of fetal alignment recommendations already provided by the fetal alignment recommendation model 116. Alternatively, the fetal alignment recommendation manager 106 may send another request to the fetal alignment recommendation model 116 for one or more fetal alignment recommendations.

FIG. 3C is a diagram of the example monitoring UI 302 and example recommendation UI 304C for fetal alignment recommendations, according to one embodiment of the present disclosure. In this example, the recommendation UI 304C includes a pictorial representation of the “Side Lying Release,” described with respect to FIG. 3B. As stated previously, the fetal alignment recommendation manager 106 may populate the UI 304C in response to a user selection on recommendation UI 304B. Although this example includes a pictorial representation of the fetal alignment recommendation, as stated previously, the fetal alignment recommendation media 110 used to populate the recommendation UI 304C may include video and/or a video and audio demonstration of the fetal alignment recommendation. Accordingly, the UI 304C may include controls for playing, rewinding, fast-forwarding the video, and the like. Additionally, the recommendation UI 304C includes a return button 312. Accordingly, once the healthcare professional and/or expectant mother has viewed the fetal alignment recommendation media 110, the healthcare professional may return to the recommendation UI 304B by selecting the return button 312.

FIG. 3D is a diagram of the example monitoring UI 302 and example recommendation UI 304D for fetal alignment recommendations, according to one embodiment of the present disclosure. The fetal alignment recommendation manager 106 may present the recommendation UI 304D in response to a selection of the history button 310 on the recommendation UI 304B. In response to this selection, the fetal alignment recommendation manager 106 may retrieve all fetal alignment recommendations made during this labor from the fetal alignment recommendation history 112. In this example, the fetal alignment recommendation history includes the names of the fetal alignment recommendations, the time of the recommendation, and an indicator as to whether the fetal alignment recommendation was attempted. Additionally, the recommendation UI 304D includes the correct button 306, to enable the healthcare provider to make updates to the history (e.g., whether the recommendation is attempted). While this example merely includes the recommendation, time, and attempt indicator, other fields in the fetal alignment recommendation history may also, or alternatively, be included, such as a result of the attempt, how long the position was held, and the like.

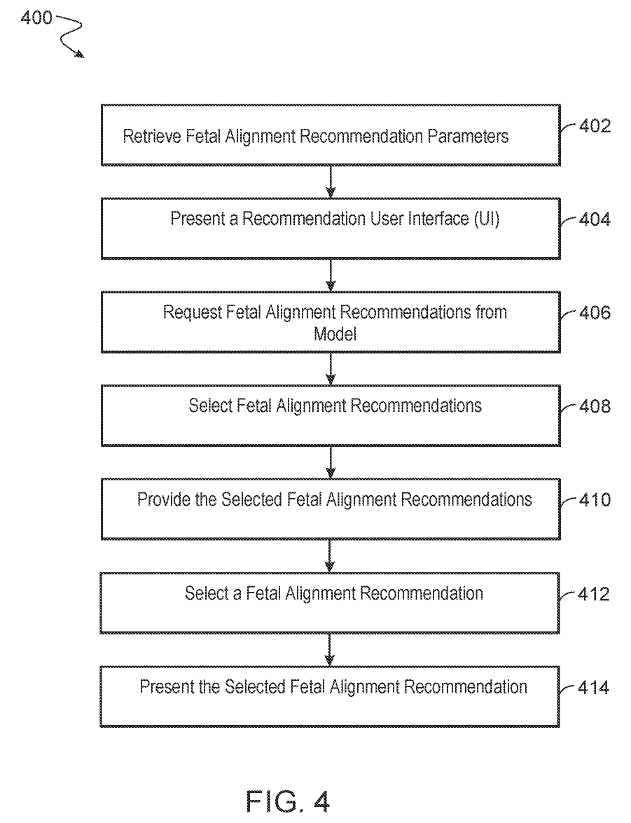

FIG. 4 is a flow chart of a method 400 for fetal alignment recommendations, according to one embodiment of the present disclosure. The fetal alignment recommendation manager 106 and fetal alignment recommendation model 116 may perform the method 400.

At operation 402, the fetal alignment recommendation manager 106 may retrieve fetal alignment recommendation parameters. As stated previously, the fetal alignment recommendation manager 106 may use the respective APIs 122, 124 of the fetal monitoring application 104 and/or clinical software applications 108 to access fetal alignment recommendation parameters. The fetal alignment recommendation parameters may include the maternal BMI, mobility limitations, gestational week, position of the fetus, presentation of the fetus, descent of the fetus, pitocin administration details, doctor or other healthcare professional instructions, and the like.

At operation 404, the fetal alignment recommendation manager 106 may present a recommendation UI on a same screen as a monitoring UI. For example, the fetal alignment recommendation manager 106 may present the recommendation UI 304A, described with respect to FIG. 3A as a pop-up window below the strip of the waveform and vitals data on the monitoring UI 118. In some embodiments, the fetal alignment recommendation manager 106 may pre-populate the fields of the recommendation UI 304A with the fetal alignment recommendation parameters retrieved at operation 402. Alternatively, the fetal alignment recommendation manager 106 may provide the recommendation UI 304A without populating the fields, thus allowing a healthcare professional or other clinician to populate the fields manually (e.g., with a keyboard and mouse). In some embodiments, the fetal alignment recommendation manager 106 may pre-populate some of the fields, and leave other fields blank, depending on what fetal alignment recommendation parameters the fetal alignment recommendation manager 106 can access.

At operation 406, the fetal alignment recommendation manager 106 may request fetal alignment recommendations from the fetal alignment recommendation model 116. In some embodiments, the healthcare professional or other clinician may request a fetal alignment recommendation by selecting the request button 308. In response, the fetal alignment recommendation manager 106 may send a request to the fetal alignment recommendation model 116 with the fetal alignment recommendation parameters, and the fetal alignment recommendation history 112 for this labor.

At operation 408, the fetal alignment recommendation model 116 may select one or more fetal alignment recommendations based on the fetal alignment recommendation parameters and the fetal alignment recommendation history 112. According to some embodiments of the present disclosure, the fetal alignment recommendation model 116 may select fetal alignment recommendations that the model determines are more likely to produce a positive result and/or positive labor outcome.

At operation 410, the fetal alignment recommendation model 116 may provide the selected fetal alignment recommendations for the fetal alignment recommendation manager. As stated previously, the fetal alignment recommendation model 116 may be trained to select multiple fetal alignment recommendations based on the fetal alignment recommendation parameters and fetal alignment recommendation history. Additionally, the fetal alignment recommendation model 116 may provide links, keys, or other identifiers, corresponding to the recommendations in the fetal alignment recommendation database 114, and/or links to fetal alignment recommendation media 110 corresponding to the recommendations.

At operation 412, the fetal alignment recommendation manager 106 may select a fetal alignment recommendation. As stated previously, the fetal alignment recommendation manager may randomly select a fetal alignment recommendation from the recommendations provided by the fetal alignment recommendation model.

At operation 414, the fetal alignment recommendation manager 106 may present the selected fetal alignment recommendation. Presenting the selected fetal alignment recommendation may involve presenting a name, and textual description, of the fetal alignment recommendation. According to some embodiments of the present disclosure, the fetal alignment recommendation manager 106 may retrieve this data from the fetal alignment recommendation database 114. Additionally, presenting the selected fetal alignment recommendation may involve retrieving fetal alignment recommendation media 110, and presenting the media on a recommendation UI (e.g., recommendation UI 304C).

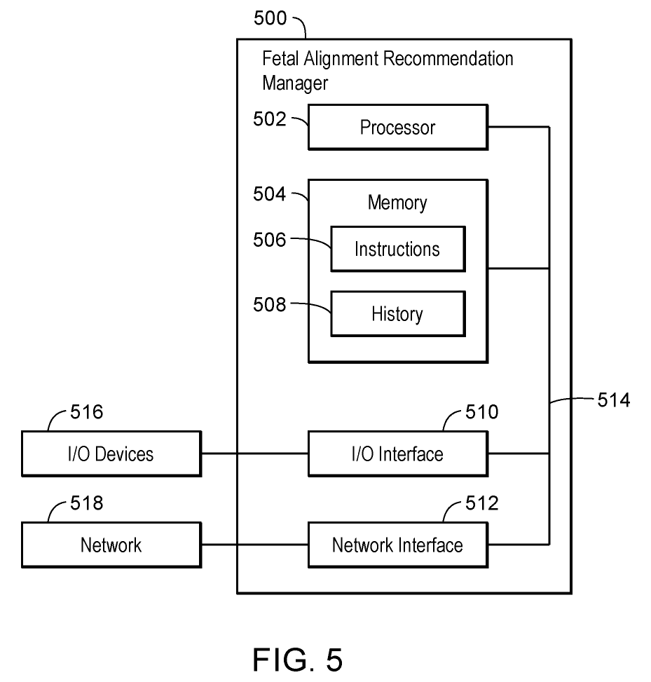

FIG. 5 is an exemplary fetal alignment recommendation manager 500, according to one embodiment of the present disclosure. The example fetal alignment recommendation manager 500 may make fetal alignment recommendations for an expectant mother during labor as described with respect to FIGS. 1, 2, 3A-3D and 4. In this example, the fetal alignment recommendation manager 500 includes a processor 502, memory 504, input-output (I/O) interface 510, and network interface 512, which may be connected by an interconnect 514. The processor 502 may be a computer processing circuit (e.g., a central processing unit (CPU)) that retrieves and executes programming instructions 506 stored in the memory 504 to perform the functionality described herein. The interconnect 514 may move data, such as programming instructions, between the processor 502, memory 504, I/O interface 510, and network interface 512. The interconnect 514 may include one or more buses.

The memory 504 may be a computer memory or storage device, including volatile memory, such as a random access memory (RAM) device (e.g., static RAM, dynamic RAM, and the like), non-volatile memory, such as a hard disk drive, solid state device (SSD), removable memory cards, optical storage, flash memory devices, and the like. In some examples, the memory 504 may include volatile and non-volatile memory devices. Further, the memory 504 may store instructions 506, and history 508. The instructions 506 may perform the techniques and/or functionality described with respect to FIGS. 1, 2, 3A-3D and 4. Further, the history 508 may be similar to the fetal alignment recommendation history 112, described with respect to FIG. 1.

Additionally, the fetal alignment recommendation manager 500 may be in electronic communication with I/O devices 516 through the I/O interface 510, and with a network 518 through the network interface 512. The I/O devices 516 may capture inputs and provide outputs as described herein. The network 518 may be an electronic communication network, such as a local area network, wide area network, and the like, for processing communications between the fetal alignment recommendation manager 500 and the machine learning models described herein. In some examples, the network 518 may be wired, wireless (e.g., wi-fi, Bluetooth, or cellular), or some other computer communication network.

In some embodiments, the fetal alignment recommendation manager 500 may be a server computer, virtual machine, cloud service, or similar device without a user interface but which receives requests from other computer systems having one or more user interfaces. Further, in some embodiments, the fetal alignment recommendation manager 500 may be a portable computer, laptop, tablet computer, pocket computer, telephone, smart phone, or the like.

An example system includes a processor and a memory. The memory includes instructions executable by the processor to generate fetal alignment recommendations by querying a database of fetal alignment recommendations. Further, querying is based on maternal health parameters for an expectant mother in labor, fetal health parameters for a fetus of the expectant mother, a position of the fetus, a presentation of the fetus, and a descent of the fetus. Additionally, the instructions are executable by the processor to randomly selecting a recommendation from multiple fetal alignment recommendations, and present the recommendation in view of a user interface of a fetal monitoring application.

In one example, querying includes using a machine learning model trained to provide the first plurality of fetal alignment recommendations based on the plurality of maternal health parameters, the plurality of fetal health parameters, the position of the fetus, the presentation of the fetus, and the descent of the fetus.

In another example, the instructions are executable by the processor to remove one or more previous fetal alignment recommendations that the expectant mother has attempted before randomly selecting the recommendation.

In another example, querying is further based on a result of implementing the one or more previous fetal alignment recommendations.

In another example, querying is further based on an instruction from a healthcare provider for the expectant mother.

In another example, querying is further based on a position of a placenta.

In another example, the fetal monitoring application determines the plurality of maternal health parameters and the plurality of fetal health parameters.

In another example, the instructions are executable by the processor to identify a media source that provides a visual representation of the recommendation, and provide the visual representation by accessing the media source, and displaying the visual representation in view of the user interface of the fetal monitoring application.

An example method includes generating fetal alignment recommendations by querying a database of fetal alignment recommendations. Querying is based on maternal health parameters for an expectant mother in labor, fetal health parameters for a fetus of the expectant mother, a position of the fetus, a presentation of the fetus, and a descent of the fetus. The method also includes generating remaining fetal alignment recommendations by removing, from the generated fetal alignment recommendations, one or more previous fetal alignment recommendations that the expectant mother has attempted. Additionally, the method includes randomly selecting a recommendation from the remaining fetal alignment recommendations. Further, the method includes presenting the recommendation in view of a user interface of a fetal monitoring application.

In one example, querying involves using a machine learning model trained to provide the fetal alignment recommendations based on the maternal health parameters, the fetal health parameters, the position of the fetus, the presentation of the fetus, and the descent of the fetus.

In another example, querying is further based on an instruction from a healthcare provider for the expectant mother.

In another example, querying is further based on a position of a placenta.

In another example, querying is further based on a result of implementing the one or more previous fetal alignment recommendations.

In another example, the fetal monitoring application determines the maternal health parameters and the fetal health parameters.

Another method includes identifying a media source that provides a visual representation of the recommendation. Additionally, the method includes providing the visual representation by accessing the media source, and displaying the visual representation in view of the user interface of the fetal monitoring application.

An example computer readable medium includes instructions executable by a processor to generate fetal alignment recommendations by querying a database of fetal alignment recommendations. Querying is based on maternal health parameters for an expectant mother in labor, fetal health parameters for a fetus of the expectant mother, a position of the fetus, a presentation of the fetus, and a descent of the fetus. Additionally, the instructions are executable by the processor to generate remaining fetal alignment recommendations by removing, from the fetal alignment recommendations, one or more previous fetal alignment recommendations that the expectant mother has attempted. Further, the instructions are executable by the processor to randomly select a recommendation from the remaining fetal alignment recommendations, and present the recommendation in view of a user interface of a fetal monitoring application.

In one example, querying includes using a machine learning model trained to provide the fetal alignment recommendations based on the maternal health parameters, the fetal health parameters, the position of the fetus, the presentation of the fetus, and the descent of the fetus.

In another example, querying is further based on a result of implementing the one or more previous fetal alignment recommendations.

In another example, the fetal monitoring application determines the maternal health parameters and the fetal health parameters.

In another example, the instructions are executable by the processor to identify a media source that provides a visual representation of the recommendation, and provide the visual representation by accessing the media source, and displaying the visual representation in view of the user interface of the fetal monitoring application.

As used herein, the term, mechanism, can encompass hardware, software, firmware, or any suitable combination thereof. In some embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

As used herein, the term, mechanism, can encompass hardware, software, firmware, or any suitable combination thereof. In some embodiments, any suitable computer readable media can be used for storing instructions for performing functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A system, comprising:
a processor; and
a memory device comprising instructions that are executable by the processor to:
generate a first plurality of fetal alignment recommendations by querying a database comprising a second plurality of fetal alignment recommendations, wherein querying is based on a plurality of maternal health parameters for an expectant mother in labor, a plurality of fetal health parameters for a fetus of the expectant mother, a position of the fetus, a presentation of the fetus, and a descent of the fetus;
randomly select a recommendation from the plurality of fetal alignment recommendations; and
present the recommendation in view of a user interface of a fetal monitoring application.

2. The system of claim 1, wherein querying comprises using a machine learning model trained to provide the first plurality of fetal alignment recommendations based on the plurality of maternal health parameters, the plurality of fetal health parameters, the position of the fetus, the presentation of the fetus, and the descent of the fetus.

3. The system of claim 1, the instructions being executable by the processor to remove one or more previous fetal alignment recommendations that the expectant mother has attempted before randomly selecting the recommendation.

4. The system of claim 3, wherein querying is further based on a result of implementing the one or more previous fetal alignment recommendations.

5. The system of claim 1, wherein querying is further based on an instruction from a healthcare provider for the expectant mother.

6. The system of claim 1, wherein querying is further based on a position of a placenta.

7. The system of claim 1, the fetal monitoring application determining the plurality of maternal health parameters and the plurality of fetal health parameters.

8. The system of claim 1, the instructions being executable by the processor to:
identify a media source that provides a visual representation of the recommendation; and
provide the visual representation by:
accessing the media source; and
displaying the visual representation in view of the user interface of the fetal monitoring application.

9. A method, comprising:
generating a first plurality of fetal alignment recommendations by querying a database comprising a second plurality of fetal alignment recommendations, wherein querying is based on a plurality of maternal health parameters for an expectant mother in labor, a plurality of fetal health parameters for a fetus of the expectant mother, a position of the fetus, a presentation of the fetus, and a descent of the fetus;
generating a remaining plurality of fetal alignment recommendations by removing, from the first plurality of fetal alignment recommendations, one or more previous fetal alignment recommendations that the expectant mother has attempted;
randomly selecting a recommendation from the remaining plurality of fetal alignment recommendations; and
presenting the recommendation in view of a user interface of a fetal monitoring application.

10. The method of claim 9, wherein querying comprises using a machine learning model trained to provide the first plurality of fetal alignment recommendations based on the plurality of maternal health parameters, the plurality of fetal health parameters, the position of the fetus, the presentation of the fetus, and the descent of the fetus.

11. The method of claim 9, wherein querying is further based on an instruction from a healthcare provider for the expectant mother.

12. The method of claim 9, wherein querying is further based on a position of a placenta.

13. The method of claim 9, wherein querying is further based on a result of implementing the one or more previous fetal alignment recommendations.

14. The method of claim 9, the fetal monitoring application determining the plurality of maternal health parameters and the plurality of fetal health parameters.

15. The method of claim 9, comprising:
identifying a media source that provides a visual representation of the recommendation; and
providing the visual representation by:
accessing the media source; and
displaying the visual representation in view of the user interface of the fetal monitoring application.

16. A computer readable medium comprising instructions executable by a processor to:
generate a first plurality of fetal alignment recommendations by querying a database comprising a second plurality of fetal alignment recommendations, wherein querying is based on a plurality of maternal health parameters for an expectant mother in labor, a plurality of fetal health parameters for a fetus of the expectant mother, a position of the fetus, a presentation of the fetus, and a descent of the fetus;
generate a remaining plurality of fetal alignment recommendations by removing, from the first plurality of fetal alignment recommendations, one or more previous fetal alignment recommendations that the expectant mother has attempted;
randomly select a recommendation from the remaining plurality of fetal alignment recommendations; and
present the recommendation in view of a user interface of a fetal monitoring application.

17. The computer readable medium of claim 16, wherein querying comprises using a machine learning model trained to provide the first plurality of fetal alignment recommendations based on the plurality of maternal health parameters, the plurality of fetal health parameters, the position of the fetus, the presentation of the fetus, and the descent of the fetus.

18. The computer readable medium of claim 16, wherein querying is further based on a result of implementing the one or more previous fetal alignment recommendations.

19. The computer readable medium of claim 16, the fetal monitoring application determining the plurality of maternal health parameters and the plurality of fetal health parameters.

20. The computer readable medium of claim 16, the instructions being executable by the processor to:
identify a media source that provides a visual representation of the recommendation; and
provide the visual representation by:
accessing the media source; and
displaying the visual representation in view of the user interface of the fetal monitoring application.
